(19) United States
(12) Patent Application Publication
(10) Pub. No.: US 2009/0081310 A1
(43) Pub. Date: Mar. 26, 2009

(54) METHOD FOR REMEDIATING A STRUCTURE CONTAMINATED WITH MOLD

(76) Inventor: John Y. Mason, Slingerlands, NY (US)

Correspondence Address: HESLIN ROTHENBERG FARLEY & MESITI PC, 5 COLUMBIA CIRCLE, ALBANY, NY, 12203, US

(21) Appl. No.: 11/576,498

(22) PCT Filed: Sep. 30, 2005
(86) PCT No.: PCT/US2005/035319

§ 371 Date: Dec. 9, 2008

Related U.S. Application Data
(60) Provisional application No. 60/615,382, filed on Oct. 1, 2004.

(51) Int. Cl. A01N 59/00; A01P 1/00

(52) U.S. Cl. 424/600

(57) ABSTRACT

The invention provides methods for using chlorine dioxide gas (100) for the remediation of mold contaminated building spaces (400), heating ventilation and air conditioning systems, vehicles, office spaces, process equipment, files, documents, mail, mail processing equipment, industrial process equipment and consumer related items under safe conditions.

### BACKGROUND OF THE INVENTION

About 1,000 species of mold can be found in the United States with more than 100,000 known species worldwide. They can damage the structure of wood framed buildings, destroy furnishings, and cause numerous deleterious health effects, including allergic reactions. The onset of allergic reactions to mold can be either immediate or delayed. Allergic responses include hay fever-type symptoms such as runny nose and red eyes.

Molds may also cause localized skin or mucosal infections but, in general, do not cause systemic infections in humans, except for persons with impaired immunity, AIDS, uncontrolled diabetes, or those taking immune suppressive drugs. Molds can also cause asthma attacks in some individuals who are allergic to mold. In addition, exposure to mold can irritate the eyes, skin, nose and throat in certain individuals.

Stachybotrys atra, now known as Stachybotrys chartarum (SC), is a destructive mold species that was implicated as the source of mold infestation in several New York City buildings in the early 1990's. Many other dangerous fungi have also been identified as sources of building contamination, e.g. species of Aspergillus, Penicillium, Fusarium, Trichoderma, and Memnoniella, all of which can produce potent mycotoxins, some of which are identical to compounds produced by SC.

Mycotoxins are fungal metabolites that have been identified as toxic agents. Two mold-produced toxins (aflatoxins and ochratoxin A) have been classified by the National Toxicology Program as human carcinogens.

Currently, there are no federal standards or recommendations, (e.g., OSHA, NIOSH, EPA) for airborne concentrations of mold or mold spores. Scientific research on the relationship between mold exposures and health effects is ongoing.

Since mold requires water to grow, a first step in remediating mold damage is to locate the source of moisture. e.g. leaks, uncontrolled humidity, etc. Clean-up efforts normally involve removal of badly damaged materials, e.g., carpets, ceiling tiles and the like, followed by surface cleanup and thorough drying.

Surface cleanup may typically be achieved by one of the following techniques: wet vacuum using sufficient water in the tank to prevent the spread of spores, damp wipe using a detergent and water solution, and HEPA vacuum. Porous materials are nearly impossible to clean in this manner and will have to be discarded. Bleach and disinfectants are generally not recommended.

There remains a need for an effective means of remediating mold in enclosed spaces particularly large structures, particularly where there is hidden mold contamination behind wallpaper, wallboard and the like. There is also no currently effective means for remediating mold-contaminated porous materials on a large scale.

Moreover, there is currently no effective means of destroying the allergenic properties of killed mold spores existing within contaminated structures on a large scale, nor is there any effective means of dealing with their mycotoxins.

### SUMMARY OF THE INVENTION

The present invention provides methods for the effective large-scale use of chlorine dioxide gas to allow for gaseous penetration of contents included within a large enclosed volume requiring mold remediation in an environmentally safe manner. The present invention provides a method comprising the steps of:

1. generating chlorine dioxide gas that is preferably free of chlorine gas and other contaminants,

2. introducing the chlorine dioxide gas into the volume requiring mold remediation,

3. distributing the introduced chlorine dioxide gas in said volume,

4. maintaining the chlorine dioxide gas within said volume at a concentration and for a sufficient duration permitting gaseous penetration of included contents as required for mold remediation, and

5. optionally, providing post-remediation testing (air monitoring and surface swabs) for mold contamination.

### BRIEF DESCRIPTION OF THE DRAWING

FIG. 1 is a schematic illustration of one embodiment of the present invention.

### DESCRIPTION OF A PREFERRED EMBODIMENT

In one embodiment, the present invention provides a process comprising producing chlorine dioxide by using an apparatus such as a chlorine dioxide generator, e.g. as disclosed and claimed in U.S. Pat. No. 6,468,479, the disclosure of which is incorporated herein by reference. The chlorine dioxide is generated either directly as a gas or more preferably as an aqueous (or other suitable liquid carrier) chlorine dioxide mixture. The generator is preferably run using an excess of sodium chlorite to reduce the possibility of generating chlorine gas as an impurity.

The carrier liquid in the generator is preferably water. In an aqueous solution, chlorine dioxide solution equilibrium partial pressure is optimally kept below about 26,000 ppmᵥ (corrected for standard temperature and pressure).

The generated chlorine dioxide is transferred directly, or alternatively, indirectly via a storage tank, to a high gas:liquid ratio emitter. In one preferred embodiment, the emitter is an apparatus such as a gas/liquid contactor having a high efficiency mist eliminator and very low liquid/gas rates. In one embodiment, the emitter is an apparatus such as a stripper.

The emitter is operated to maintain the gaseous chlorine dioxide concentration substantially below the explosion limit of chlorine dioxide in the air. Prior to generation of the chlorine dioxide, the emitters may be used with water alone to raise the relative humidity in the volume requiring remediation, with adjustment of the temperature. Alternatively, the humidification and remediation can be done simultaneously using the same apparatus by the appropriate adjustment in the temperature of chlorine dioxide solution. This pre-humidification may be helpful in swelling the spore coats of resistant molds and may aid in remediating particularly recalcitrant species. Control of humidity level during remediation may also aid in gaseous penetration of some porous surfaces.

The treatment is conducted in reduced illumination, preferably substantially dark, to minimize the decomposition of chlorine dioxide to chlorine. The process is monitored with the use of an infrared camera or similar device.

If the space to be remediated contains materials that are potentially susceptible to corrosion, the chlorine dioxide should be of the highest possible purity. Specifically, chlorine gas should be present in the introduced chlorine dioxide gas at a level less than about 5%, preferably less than about 0.5%.

Once the desired relative humidity and temperature are attained, then the variable generation rate of chlorine dioxide gas is initiated. The initial rate is high to provide sufficient chlorine dioxide to penetrate the various surfaces demands within the volume requiring remediation. This rate is predetermined to accommodate the surface demand as well as to provide the initial charge of the volume requiring remediation to a predetermined chlorine dioxide residual level. The chlorine dioxide generation rate is then reduced appropriately to maintain the predetermined chlorine dioxide concentration in the air of the volume requiring remediation for a predetermined time. This can be achieved by a number of means, such as lowering the concentration of chlorine dioxide in the solution that is fed to the emitter, or lowering the flow rate of the chlorine dioxide solution to the emitter.

The chlorine dioxide gas concentration is determined to compensate for the decay or loss rate from the volume requiring remediation. The volume requiring remediation is preferably to be at slightly negative pressure to areas outside of it and efforts are made to seal off the volume through the use of strippable sealant, such as foam that sets up hard. Once the required time weighted average concentration and contact time are attained, then the generation of chlorine dioxide is stopped.

The generator, storage and emitter are then purged with fresh water. Once this is complete, the water may be injected with an alkalizing and dechlorinating agent or other functional chemistry (e.g., ascorbic acid), that will scrub the chlorine dioxide. This scrubbing solution is then fed to the emitter and with the blowers still in operation, the emitter begins to scrub chlorine dioxide out of the environmental air composition within the said volume that has been remediated. This process is continued until the environmental air composition within the volume that has been remediated is returned to acceptable limits for reopening to the exterior environment and rehabitation.

The emitters can be located inside or outside of the volume requiring remediation. However, it is highly preferred to locate the emitter inside the volume requiring remediation, since then no contaminated air is allowed to leave the volume requiring remediation.

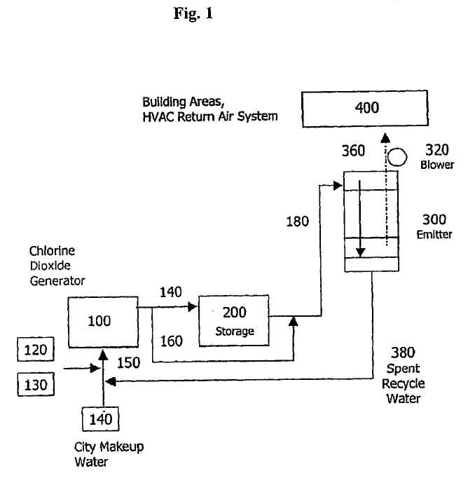

The present invention can be further understood by reference to FIG. 1, which schematically illustrates one embodiment of the invention. The invention is practiced using a chlorine dioxide gas generator 100 that provides chlorine dioxide dissolved in water 180 directly 160, or alternatively, indirectly 140 via storage 200, to a emitter 300 comprising a blower 320 that produces chlorine dioxide gas in air 360 for treatment of building areas and/or a heating, ventilation and air conditioning (HVAC) return air system of a building 400

The chlorine dioxide generator 100 receives an input 150 that is a mixture of city makeup water 140 and chemicals 120 or 130. Suitable mixing means are used to combine the city makeup water 140 and chemicals 120 or 130. Metering means are used to regulate the amounts and proportions of the city makeup water 140 and chemicals 120 or 130 that are combined. In the optional climatizing stage of the process, water alone is provided to the input 150 of the chlorine dioxide generator 100, in order to adjust the relative humidity of the volume to be treated. In the second stage of the process, chlorine dioxide precursor chemicals 120 are combined with the city makeup water 140. Several chemical means of generating chlorine dioxide and their corresponding chlorine dioxide precursor chemicals are known in the art, and the choice of suitable means and chemicals is within the abilities of the skilled artisan. Exemplary chemical means of generating chlorine dioxide are disclosed in U.S. Pat. Nos. 4,689,169 (Mason et al.), 5,204,081 (Mason et al.), 5,227,306 (Eltomi et al.), 5,258,171 (Eltomi et al.), 5,965,004 (Cowley et al.), 6,468,479 (Mason et al.) and 6,645,457 (Mason et al.) the teachings of which are hereby incorporated by reference.

The output of the chlorine dioxide generator 100 can be routed directly 160 to provide chlorine dioxide dissolved in water 180 to the emitter 300. Alternatively, the output of the chlorine dioxide generator can be routed 140 to a storage means 200, from which chlorine dioxide dissolved in water 180 can be routed to the emitter 300.

The emitter 300 removes chlorine dioxide from the water and delivers chlorine dioxide in air 360 by duct means to the volume to be remediated, in general, building areas and/or a HVAC return air system; “Duct means” includes, but is not limited to, temporary or permanent ductwork, pipes, hoses and the like. Water 380 recovered from the emitter can be recycled and combined by mixing means with city makeup water 140 and chemicals 120 or 130 to provide input 150 to the chlorine dioxide generator 100.

In a third stage of the process, the chlorine dioxide generator 100, storage 200 and emitter 300 are flushed with water alone. During a further stage of the process, detoxification chemicals 130 are combined with water to provide the input to the chlorine dioxide generator 100.

Monitoring and controlling the dew point within the volume requiring remediation is a significant aspect. During the process of remediation, steps must be taken to avoid condensation. Therefore during the entire remediation process the atmosphere within the volume requiring remediation must be carefully controlled using space heaters or the HVAC system both to avoid over-humidification and to regulate the temperature of the chlorine dioxide solution fed to the emitter. Failure to control these factors can lead to spot damage as well as a higher use of chlorine dioxide.

As used herein, “CT” equals the time weighted average chlorine dioxide concentration multiplied by the exposure time in hours. In a plot of chlorine dioxide concentration over exposure time in hours, the CT would equal the area under the curve. For example, if the time weighted average chlorine dioxide concentration over a 12 hour exposure period were 750 ppmᵥ, the CT would be 9000 ppmᵥ hours.

It is an aspect of this invention to minimize the chlorine dioxide concentration and CT to the extent possible to assure decontamination while avoiding damage to building contents such as electronic equipment (e.g., telephone equipment, computers, copiers, and other electronic office equipment), furnishings, and the like. Typical chlorine dioxide concentrations are in the range of 500 to 3000 ppmᵥ, and exposure times are typically about 8 to 12 hours. For mold remediation, a time averaged chlorine dioxide gas concentration in the range of about 500 to 1500 ppmᵥ over a 12 hour period has been found effective for killing mold spores and eliminating allergenic effects.

Pre-testing of contaminated materials can be done on a smaller scale to determine the effective concentration and exposure time. Test samples of contaminated material may be taken from the building prior to remediation and subjected to chlorine dioxide treatment to assess the effective concentration and exposure time.

### Example 1

An approximately 50,000 ft² mold-contaminated building (1 million cubic feet approximately) was remediated by fumigating the building with chlorine dioxide gas at a concentration within the range of 2,500 to 3,000 ppmᵥ over a period of 3 hours. Temperature was maintained at approximately 75° F. and the relative humidity was maintained at approximately 75% before and during fumigation. Carpets and other porous materials within the contaminated building were not removed prior to fumigation. The foregoing process substantially eliminated viable mold and mold spores from the facility, and substantially eliminated the biological mass from the mold growth. Porous surfaces that were black, grey and green shades prior to treatment were returned to original appearance. The mold mass was also substantially eliminated such that one to four square foot samples were required to collect 0.1 to 0.25 grams of mold residue resulting in a 99+ percent elimination in mold mass. Samples of the treated material were analyzed by PCR (polymerase chain reaction) analysis for DNA. Depending upon the initial concentration levels a 3 to 7 log reduction in DNA was observed. An industrial hygiene review of the facility found no evidence of mold contamination or residual mold alegenicity or mycotoxin presence.

### Example 2

A structure of approximately 6000 square feet consisting of three above surface floors and a basement was located that had developed significant mold growth of a variety of species due to water intrusion and appropriate environmental conditions. Personnel entering the structure exhibited immediate symptoms of allergic responses including sinus congestion, irritated upper respiratory tract, and eyes. Mold growth was visible and abundant upon walls, ceilings and other surfaces.

The temperature of the structure was raised and maintained in the range of 70 to 100° F. (21 to 38° C.) and the relative humidity was raised to a minimum of 65%. Chlorine dioxide solution was generated in a closed fluid loop by the process described in U.S. Pat. No. 6,468,479. Chlorine dioxide gas was added to the structure at such a rate as to maintain a concentration of between 600 and 1300 ppmᵥ for a 12 hour period and a total CT of over 9000 ppmᵥ hours of chlorine dioxide.

Chlorine dioxide concentration and purity were monitored by AWWA standard method 6500-ClO2-E in solution phase and a modification therewith to adapt the procedure for gas phase sampling. The structure was contained within a tent made of reinforced 16 mil PVC that was fitted to the structure and maintained under a slight negative pressure of approximately 0.05 inches of water column. The removed air was scrubbed through activated carbon filters. The maximum chlorine dioxide concentration detected exterior to the containment tent was less than 2 parts per billion, well within the maximum exposure guidelines established.

Following treatment mold viability was evaluated in the structure. Substantially 100% percent of all mold and mold spores were rendered non viable, including mold underneath wallpaper and the exterior paper coating of wallboard. Greater extent of kill determination was not possible due to the environmental prevalence of mold spores. Visual inspection of the treated mold demonstrated a color change from black to a light tan coloration. The non viable mold was very easy to remove. Mold kill was observed on all surfaces and beneath wall paper. No personnel entering the facility post treatment reported allergenic responses even after direct exposure to the residual biological matter.

The present invention is not to be limited in scope by the specific embodiments described herein, but by the appended claims. The described embodiments are intended as illustrations of individual aspects of the invention, and functionally equivalent methods and components are within the scope of the invention. Indeed, various modifications of the invention, in addition to those shown and described herein will become apparent to those skilled in the art from the foregoing description and accompanying drawing. Such modifications are intended to fall within the scope of the appended claims.

We claim:

1. A method suitable for remediating a large enclosed mold contaminated volume and contents requiring remediation and restoring habitability, comprising the steps of: generating chlorine dioxide gas; introducing the chlorine dioxide gas into the volume requiring remediation; distributing the introduced chlorine dioxide gas in the volume requiring remediation; and maintaining a concentration of the chlorine dioxide gas within the volume requiring remediation under environmentally safe conditions at a level and duration permitting gaseous penetration of included contents as required for remediation, thereby remediating the large enclosed volume and contents and restoring habitability.

2. The method of claim 1, wherein the time weighted average concentration of the chlorine dioxide gas and the exposure time are selected so that the mold contamination is rendered substantially non-allergenic.

3. The method of claim 1 wherein the building is enclosed within a substantially light impervious tent while undergoing remediation so as to avoid light-induced degradation of the introduced chlorine dioxide gas.

4. The method of claim 3, wherein the tent is substantially impervious to gas.

5. The method of claim 1 wherein the time weighted average chlorine dioxide gas concentration is maintained in the range of about 500 to 3000 ppmᵥ for a time period of about 3 to 12 hours.

6. The method of claim 1, wherein at least a portion of the mold to be treated is underneath wallpaper.

7. A building structure which has been restored to habitability following mold contamination, using a method suitable for remediating a large enclosed mold contaminated volume and contents requiring remediation and restoring habitability, comprising the steps of: generating chlorine dioxide gas; introducing the chlorine dioxide gas into the volume requiring remediation; distributing the introduced chlorine dioxide gas in the volume requiring remediation; and maintaining a concentration of the chlorine dioxide gas within the volume requiring remediation under environmentally safe conditions at a level and duration permitting gaseous penetration of included contents as required for remediation, thereby remediating the large enclosed volume and contents and restoring habitability.

8. The building structure of claim 7, wherein the time weighted average concentration of the chlorine dioxide gas and the exposure time are selected so that the mold contamination is rendered substantially non-allergenic.

9. The building structure of claim 7, wherein the building is enclosed within a substantially light impervious tent while undergoing remediation so as to avoid light-induced degradation of the introduced chlorine dioxide gas.

10. The building structure of claim 9, wherein the tent is substantially impervious to gas.

11. The building structure of claim 7, wherein the time weighted average chlorine dioxide gas concentration is maintained in the range of about 500 to 3000 ppmᵥ for a time period of about 3 to 12 hours.

12. The building structure of claim 7, wherein at least a portion of the mold to be treated is underneath wallpaper.
